(19) United States
(12) Patent Application Publication
(10) Pub. No.: US 2013/0302266 A1
(43) Pub. Date: Nov. 14, 2013

(54) COSMETIC COMPOSITION COMPRISING AT LEAST ONE CATIONIC POLYMER AND AT LEAST TWO CATIONIC SURFACTANTS

(71) Applicants: Claire Bourdin, Levallois Perret (FR); Audrey Gilles, Paris (FR); Cécile Bebot, Clichy (FR)

(72) Inventors: Claire Bourdin, Levallois Perret (FR); Audrey Gilles, Paris (FR); Cécile Bebot, Clichy (FR)

(73) Assignee: L'OREAL, Paris (FR)

(21) Appl. No.: 13/821,043

(22) PCT Filed: Sep. 6, 2011
(86) PCT No.: PCT/EP11/65404

§ 371 Date: May 20, 2013

Related U.S. Application Data
(60) Provisional application No. 61/383,788, filed on Sep. 17, 2010.

(30) Foreign Application Priority Data
Sep. 6, 2010 (FR) 1057059

(51) Int. Cl. A61K 8/84; A61K 8/34; A61Q 5/12; A61K 8/73

(52) U.S. Cl. CPC A61K 8/84; A61K 8/731; A61K 8/345; A61Q 5/12
USPC 424/70.13

(57) ABSTRACT

The invention relates to a cosmetic composition comprising, in a cosmetically acceptable medium: (i) one or more cationic polymers, at least one cationic polymer being a dimethyldiallylammonium chloride homopolymer, (ii) two or more than two cationic surfactants, (iii) one or more polysaccharide thickeners, (iv) one or more polyols with a molecular weight of less than 1000.

A subject-matter of the present invention is a cosmetic composition comprising at least one cationic polymer and at least two surfactants.

It is well known that hair which has been sensitized, embrittled or indeed even damaged under the action of atmospheric agents or of mechanical and chemical treatments, such as dyeing operations, bleaching operations and/or perming operations, is often difficult to disentangle and to style and lacks softness. It also lacks sheen, the surface of the hair being detrimentally affected and sending back the light in a less homogeneous way.

The use has already been recommended, in compositions for the care of keratinous substances, such as the hair, of conditioning agents, in particular silicones, for facilitating the disentangling of the hair and for conferring softness and suppleness thereon. In the case in particular of leave-in care products, for the smoothness and sheen, it is generally silicone gums, in solution in volatile organic solvents, which are used.

However, the cosmetic advantages contributed by these conditioning agents are unfortunately also accompanied, on dry hair, by a greasy feel and a straggly visual appearance, which may be accompanied by faster regreasing of the hair between two shampooing operations.

Furthermore, the volatile organic solvents used to dissolve the silicone conditioning agents (liquid fatty ester, hydrocarbon oil of isododecane or isohexadecane type and/or silicone oil of cyclomethicone type) do not make it possible to overcome the abovementioned disadvantages.

In addition, in the case of anhydrous products in particular, these volatile solvents are present at a high concentration in the product and they have the disadvantage of having a sometimes disputed environmental profile.

There thus exists a need to have available more effective conditioning compositions.

In some cases, clear compositions are also sought for.

The Applicant Company has discovered, surprisingly, that compositions comprising at least one cationic polymer and at least two specific surfactants make it possible to solve the problems touched on above.

More specifically, a subject-matter of the invention is a cosmetic composition comprising, in a cosmetically acceptable medium:

(i) one or more cationic polymers,

(ii) two or more than two cationic surfactants,

(iii) one or more polysaccharide thickeners,

(iv) one or more polyols with a molecular weight of less than 1000.

The term “molecular weight” is understood to mean the molecular weight expressed in g/mol.

Such a composition is easily distributed over the hair, resulting in clean, shiny, smooth and soft hair which is easily disentangled and the good cosmetic properties of which endure over time, even after several shampooing operations.

A further subject-matter of the invention is a method for the nontherapeutic cosmetic treatment of keratinous substances, in particular the hair, comprising the application, to the said substances, of a composition as described above.

Cationic Polymer

The composition according to the invention comprises one or more cationic polymers.

The term “cationic polymer” means a polymer that is positively charged when it is contained in the composition according to the invention. This polymer may bear one or more positive permanent charges or may contain one or more cationizable functions in the composition according to the invention.

At least one of the cationic polymers of the composition is a dimethyldiallylammonium chloride homopolymer sold under the name Merquat 100 by Nalco (and its homologues of low weight-average molecular weights).

The composition according to the invention can also comprise one or more other cationic polymer(s). The other cationic polymer(s) that may be used as conditioning agents according to the present invention are preferably chosen from polymers comprising primary, secondary, tertiary and/or quaternary amine groups forming part of the polymer chain or directly attached thereto, and having a molecular weight of between 500 and about 5 000 000 and preferably between 1000 and 3 000 000.

When the conditioning agent is a cationic polymer, it is preferably chosen from those that contain units comprising primary, secondary, tertiary and/or quaternary amine groups that may either form part of the main polymer chain, or may be borne by a side substituent directly attached thereto.

Among the cationic polymers that may be mentioned more particularly are polymers of the polyamine, polyaminoamide and polyquaternary ammonium type. These are known products. They are described, for example, in French Patents 2 505 348 and 2 542 997.

Among these polymers, mention may be made of:

(1) homopolymers or copolymers that are derived from acrylic or methacrylic esters or amides and comprise at least one of the units of the following formulae:

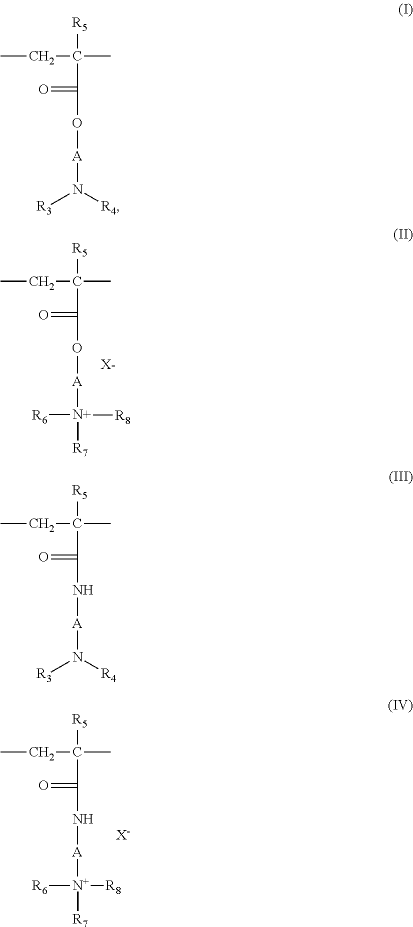

in which:

R3 and R4, which may be identical or different, represent hydrogen or an alkyl group containing from 1 to 6 carbon atoms and preferably methyl or ethyl;

R5, which may be identical or different, denote a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R6, R7 and R8, which may be identical or different, represent an alkyl group containing from 1 to 18 carbon atoms or a benzyl radical and preferably an alkyl group containing from 1 to 6 carbon atoms;

X denotes an anion derived from a mineral or organic acid, such as a methosulphate anion or a halide such as chloride or bromide.

The copolymers of the family (1) can also contain one or more units deriving from comonomers that may be chosen from the family of acrylamides, methacrylamides, diacetone acrylamides, acrylamides and methacrylamides substituted on the nitrogen with lower (C1-C4) alkyls, acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulphate or with a methyl halide, such as the product sold under the name Hercofloc by the company Hercules,
the copolymers of acrylamide and of methacryloyloxyethyltrimethylammonium chloride described, for example, in Patent Application EP-A-080 976 and sold under the name Bina Quat P 100 by the company Ciba Geigy,
the copolymer of acrylamide and of methacryloyloxyethyltrimethylammonium methosulphate sold under the name Reten by the company Hercules,
quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French Patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylam idopropyldimethylamine copolymers sold in particular under the name Styleze CC 10 by ISP,
quaternized vinylpyrrolidone/dimethylaminopropylmethacrylamide copolymers such as the product sold under the name Gafquat HS 100 by the company ISP, and
crosslinked polymers of methacryloyloxy(C1-C4)alkyltri(C1-C4)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba.

(2) Polymers formed from piperazinyl units and divalent alkylene or hydroxyalkylene radicals containing straight or branched chains, optionally interrupted with oxygen, sulphur or nitrogen atoms or with aromatic or heterocyclic rings, and also the oxidation and/or quaternization products of these polymers. Such polymers are especially described in French Patents 2 162 025 and 2 280 361.

(3) Water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are especially described in French Patents 2 252 840 and 2 368 508.

(4) Polyaminoamide derivatives resulting from the condensation of polyalkylenepolyamines with polycarboxylic acids followed by alkylation with difunctional agents. Mention may be made, for example, of adipic acid/dialkylaminohydroxyalkyldialkylenetriamine polymers in which the alkyl radical contains from 1 to 4 carbon atoms and preferably denotes methyl, ethyl or propyl. Such polymers are especially described in French Patent 1 583 363.

Among these derivatives, mention may be made more particularly of the adipic acid/dimethylaminohydroxypropyldiethylenetriam ine polymers sold under the name Cartaretine F, F4 or F8 by the company Sandoz.

(5) The polymers obtained by reaction of a polyalkylenepolyamine containing two primary amine groups and at least one secondary amine group with a dicarboxylic acid chosen from diglycolic acid and saturated aliphatic dicarboxylic acids having from 3 to 8 carbon atoms. The mole ratio between the polyalkylenepolyamine and the dicarboxylic acid is between 0.8:1 and 1.4:1; the polyaminoamide resulting therefrom is reacted with epichlorohydrin in a mole ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyldiethylenetriamine copolymer.

(6) Alkyldiallylamine or dialkyldiallylammonium cyclopolymers, such as homopolymers or copolymers comprising, as main constituent of the chain, units corresponding to the formulae (V) and (VI):

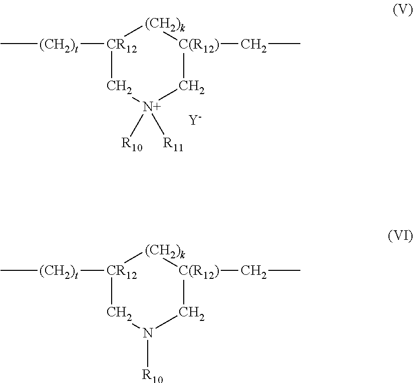

in which k and t are equal to 0 or 1, the sum k+t being equal to 1; R12 denotes a hydrogen atom or a methyl group; R10 and R11, independently of each other, denote an alkyl group having from 1 to 6 carbon atoms, a hydroxyalkyl group in which the alkyl group has preferably 1 to 5 carbon atoms, a lower (C1-C4) amidoalkyl group, or R10 and R11 may denote, jointly with the nitrogen atom to which they are attached, heterocyclic groups, such as piperidinyl or morpholinyl; Y⁻ is an anion such as bromide, chloride, acetate, borate, citrate, tartrate, bisulphate, bisulphite, sulphate or phosphate. These polymers are especially described in French Patent 2 080 759 and in its Certificate of Addition 2 190 406.

R10 and R11, independently of one another, preferably denote an alkyl group having from 1 to 4 carbon atoms.

Among the polymers defined above, mention may be made more particularly of the copolymers of diallyldimethylammonium chloride and of acrylamide, sold under the name Merquat 550.

(7) The diquaternary ammonium polymer containing repeating units corresponding to the formula:

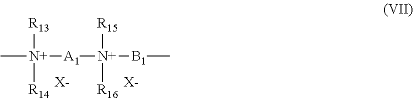

in which formula (VII):
R13, R14, R15 and R16, which may be identical or different, represent aliphatic, alicyclic or arylaliphatic radicals containing from 1 to 20 carbon atoms, or lower hydroxyalkylaliphatic radicals, or else R13, R14, R15 and R16, together or separately, constitute, with the nitrogen atoms to which they are attached, heterocycles optionally containing a second heteroatom other than the nitrogen, or else R13, R14, R15 and R16 represent a linear or branched C1-C6 alkyl radical which is substituted by a nitrile, ester, acyl, amide or —CO—O—R17-D or —CO—NH—R17-D group in which R17 is an alkylene and D is a quaternary ammonium group;
A1 and B1 represent polymethylene groups containing from 2 to 20 carbon atoms, which may be linear or branched, saturated or unsaturated, and which may contain, linked to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups, and
X⁻ denotes an anion derived from a mineral or organic acid;
A1, R13 and R15 may form, with the two nitrogen atoms to which they are attached, a piperazine ring; moreover, if A1 denotes a saturated or unsaturated, linear or branched alkylene or hydroxyalkylene radical, B1 may also denote a group (CH₂)ₙ—CO-D-OC—(CH₂)ₚ—

n and p are integers ranging from 2 to 20 approximately
in which D denotes:
a) a glycol residue of formula: —O—Z—O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—
where x and y denote an integer from 1 to 4, representing a defined and unique degree of polymerization, or any number from 1 to 4, representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;
d) a ureylene group of formula: —NH—CO—NH—;

Preferably, X⁻ is an anion such as chloride or bromide.

These polymers generally have a number-average molecular weight of between 1000 and 100 000.

Polymers of this type are described in particular in French Patents 2 320 330, 2 270 846, 2 316 271, 2 336 434 and 2 413 907 and U.S. Pat. No. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may more particularly be made of the polymers which are composed of repeat units corresponding to the formula (VIII):

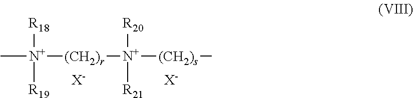

in which R18, R19, R20 and R21, which may be identical or different, denote an alkyl or hydroxyalkyl radical having from about 1 to 4 carbon atoms, r and s are integers varying from 2 to 20 approximately, and X⁻ is an anion derived from a mineral or organic acid.

One particularly preferred compound of formula (VIII) is that for which R18, R19, R20 and R21 represent a methyl radical and r=3, s=6 and X═Cl, which is called Hexadimethrine chloride according to the INCI (CTFA) nomenclature.

(8) Polyquaternary ammonium polymers composed of units of formula (IX):

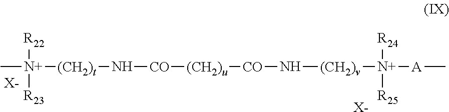

in which formula:
R22, R23, R24 and R25, which may be identical or different, represent a hydrogen atom or a methyl, ethyl, propyl, β-hydroxyethyl, β-hydroxypropyl or —CH₂CH₂(OCH₂CH₂)ₚOH radical,
in which p is equal to 0 or to an integer between 1 and 6, with the proviso that R22, R23, R24 and R25 do not simultaneously represent a hydrogen atom,
t and u, which may be identical or different, are integers between 1 and 6,
v is equal to 0 or to an integer between 1 and 34,
X⁻ denotes an anion such as a halide,
A denotes a radical of a dihalide or represents preferably —CH₂—CH₂—O—CH₂—CH₂—.

Such compounds are described especially in Patent Application EP-A-122 324.

Among these, mention may be made, for example, of the products Mirapol® A 15, Mirapol® AD1, Mirapol® AZ1 and Mirapol® 175, sold by the company Miranol.

(9) Quaternary polymers of vinylpyrrolidone and of vinylimidazole, such as, for example, the products sold under the names Luviquat® FC 905, FC 550 and FC 370 by the company BASF.

(10) Cationic polysaccharides, especially cationic celluloses and galactomannan gums.

Among cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups described in French Patent 1 492 597. These polymers are also defined in the CTFA dictionary as quaternary ammoniums of hydroxyethylcellulose that has reacted with an epoxide substituted with a trimethylammonium group.

Cationic cellulose copolymers or cellulose derivatives grafted with a water-soluble quaternary ammonium monomer are described especially in U.S. Pat. No. 4,131,576, such as hydroxyalkylcelluloses, for instance hydroxymethyl-, hydroxyethyl- or hydroxypropyl-celluloses grafted, in particular, with a methacryloyloxyethyltrimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing cationic trialkylammonium groups. Guar gums modified with a salt (e.g. chloride) of 2,3-epoxypropyltrimethylammonium are used, for example.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted to the chain. Their molecular weight may vary, for example, from 1500 to 10 000 and in particular from 2000 to 5000 approximately. Among these compounds, mention may be made especially of:
collagen hydrolysates carrying triethylammonium groups, such as the products sold under the name Quat-Pro E by the company Maybrook and referred to in the CTFA dictionary as Triethonium Hydrolyzed Collagen Ethosulfate;
collagen hydrolysates bearing trimethylammonium chloride and trimethyl-stearylammonium chloride groups, which are sold under the name Quat-Pro S by the company Maybrook and are referred to in the CTFA dictionary as Steartrimonium Hydrolyzed Collagen;
animal protein hydrolysates which bear trimethylbenzylammonium groups, such as the products sold under the name Crotein BTA by the company Croda and referred to in the CTFA dictionary as Benzyltrimonium hydrolyzed animal protein; protein hydrolysates bearing quaternary ammonium groups on the polypeptide chain, the said ammonium groups containing at least one alkyl radical having from 1 to 18 carbon atoms.

Among these protein hydrolysates, mention may be made, inter alia, of:
Croquat L, in which the quaternary ammonium groups contain a C12 alkyl group;
Croquat M, in which the quaternary ammonium groups contain C10-C18 alkyl groups;
Croquat S, in which the quaternary ammonium groups contain a C18 alkyl group;
Crotein Q, in which the quaternary ammonium groups contain at least one alkyl group having from 1 to 18 carbon atoms.

These various products are sold by the company Croda.

Other quaternized proteins or hydrolysates are, for example, those corresponding to the formula (X):

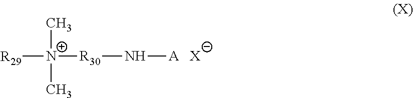

in which X⁻ is an anion of an organic or inorganic acid, A denotes a protein residue derived from collagen protein hydrolysates, R29 denotes a lipophilic group containing up to 30 carbon atoms, R30 represents an alkylene group having 1 to 6 carbon atoms. Mention may be made, for example, of the products sold by the company Inolex, under the name Lexein QX 3000, referred to in the CTFA dictionary as Cocotrimonium Collagen Hydrolysate.

Mention may also be made of quaternized plant proteins such as wheat, maize or soybean proteins: quaternized wheat proteins that may be mentioned include those sold by the company Croda under the names Hydrotriticum WQ or QM, referred to in the CTFA dictionary as Cocodimonium hydrolysed wheat protein, Hydrotriticum QL, referred to in the CTFA dictionary as Laurdimonium hydrolysed wheat protein, or else Hydrotriticum QS, referred to in the CTFA dictionary as Steardimonium hydrolysed wheat protein.

Among all the cationic polymers capable of being used in the context of the present invention, it is preferable to employ cationic cyclopolymers, in particular the dimethyldiallylammonium chloride homopolymers or copolymers sold under the names Merquat 100, Merquat 550 and Merquat S by the company Nalco, quaternary polymers of vinylpyrrolidone and of vinylimidazole, cationic polysaccharides and their mixtures.

The cationic polymer or polymers i) can be present in a content ranging from 0.01% to 15% by weight, preferably ranging from 0.1% to 10% by weight and more preferentially ranging from 0.2% to 5% by weight, with respect to the total weight of the composition of the invention.

Cationic Surfactant

The composition according to the invention comprises two or more specific cationic surfactants.

According to one alternative form, the composition comprises three specific surfactants. Generally, a cationic surfactant can carry one or more permanent positive charges or can comprise one or more functional groups which can form cations in the composition according to the invention. The cationic surfactants of the invention are those carrying one or more permanent positive charges, that is to say one or more quaternized nitrogen atoms.

Mention may in particular be made, as example of cationic surfactants which can be used in the cosmetic composition according to the invention, of:
quaternary ammonium salts, such as the quaternary ammonium salts which exhibit the following general formula (XI):

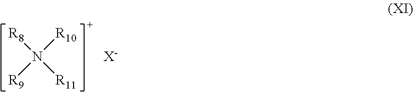

in which the R₈ to R₁₁ radicals, which can be identical or different, represent a linear or branched aliphatic radical comprising from 1 to 30 carbon atoms, or an aromatic radical, such as aryl or alkylaryl, at least one of the R₈ to R₁₁ radicals comprising from 8 to 30 carbon atoms, preferably from 12 to 24 carbon atoms. The aliphatic radicals can comprise heteroatoms, such as, in particular, oxygen, nitrogen or sulphur, and halogens.

The aliphatic radicals are, for example, chosen from alkyl, alkoxy, polyoxy(C₂-C₆)alkylene, alkylamide, (C₁₂-C₂₂)alkylamido(C₂-C₆)alkyl, (C₁₂-C₂₂)alkyl acetate or hydroxyalkyl radicals comprising approximately from 1 to 30 carbon atoms; X⁻ is an anion chosen from the group of the halides, phosphates, acetates, lactates, (C₂-C₆)alkyl sulphates, or alkyl- or alkylarylsulphonates;
imidazoline quaternary ammonium salts, such as, for example, those of following formula (XII):

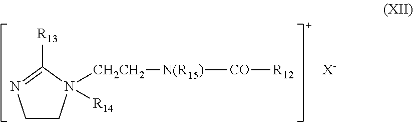

in which R₁₂ represents an alkenyl or alkyl radical comprising from 8 to 30 carbon atoms, for example derived from tallow fatty acids, R₁₃ represents a hydrogen atom, a C₁-C₄ alkyl radical or an alkenyl or alkyl radical comprising from 8 to 30 carbon atoms, R₁₄ represents a C₁-C₄ alkyl radical, R₁₅ represents a hydrogen atom or a C₁-C₄ alkyl radical and X⁻ is an anion chosen from the group of the halides, phosphates, acetates, lactates, alkyl sulphates, or alkyl- or alkylarylsulphonates. Preferably, R₁₂ and R₁₃ denote a mixture of alkenyl or alkyl radicals comprising from 12 to 21 carbon atoms, for example derived from tallow fatty acids, R₁₄ denotes a methyl radical and R₁₅ denotes a hydrogen atom. Such a product is, for example, sold under the name Rewoquat® W 75 by Rewo;
di- or triquaternary ammonium salts, in particular of formula (XIII):

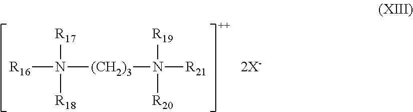

in which R₁₆ denotes an alkyl radical comprising approximately from 16 to 30 carbon atoms which is optionally hydroxylated and/or interrupted by one or more oxygen atoms, R₁₇ is chosen from hydrogen or an alkyl radical comprising from 1 to 4 carbon atoms or an (R₁₆ₐ)(R₁₇ₐ)(R₁₈ₐ)N⁺(CH₂)₃— group, R₁₆ₐ, R₁₇ₐ, R₁₈ₐ, R₁₈, R₁₉, R₂₀ and R₂₁, which are identical or different, are chosen from hydrogen or an alkyl radical comprising from 1 to 4 carbon atoms and X⁻ is an anion chosen from the group of the halides, acetates, phosphates, nitrates and (C₁-C₄)alkyl sulphates. Such compounds are, for example, Finquat CT-P, provided by Finetex (Quaternium 89), or Finquat CT, provided by Finetex (Quaternium 75),
quaternary ammonium salts comprising at least one ester functional group, such as those of following formula (XIV):

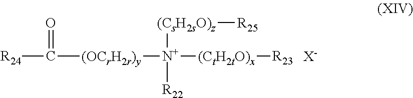

in which:
R₂₂ is chosen from C₁-C₆ alkyl and C₁-C₆ hydroxyalkyl or dihydroxyalkyl radicals;
R₂₃ is chosen from:
the

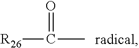

radicals R₂₇, which are linear or branched, saturated or unsaturated C₁-C₂₂ hydrocarbon-based radicals,
a hydrogen atom,

R₂₅ is chosen from:
the

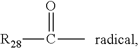

radicals R₂₉, which are linear or branched, saturated or unsaturated C₁-C₆ hydrocarbon-based radicals,
a hydrogen atom,

R₂₄, R₂₆ and R₂₈, which may be identical or different, are chosen from linear or branched, saturated or unsaturated C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₂₃ denotes R₂₇ and that when z is 0, then R₂₅ denotes R₂₉.

The alkyl radicals R₂₂ may be linear or branched, but more particularly linear.

Preferably, R₂₂ denotes a methyl, ethyl, hydroxyethyl or dihydroxypropyl radical and more particularly a methyl or ethyl radical.

Advantageously, the sum x+y+z has a value from 1 to 10.

When R₂₃ is a hydrocarbon-based radical R₂₇, it may be long and may contain from 12 to 22 carbon atoms, or may be short and may contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

Preferably, r, s and t, which are identical or different, have the value 2 or 3 and more particularly still are equal to 2.

The anion X⁻ is preferably a halide (chloride, bromide or iodide) or an alkyl sulphate, more particularly methyl sulphate. However, use may be made of methanesulphonate, phosphate, nitrate, tosylate, an anion derived from an organic acid, such as acetate or lactate, or any other anion compatible with the ammonium comprising an ester functional group.

The anion X⁻ is more particularly still chloride or methyl sulphate.

Use is more particularly made, in the composition according to the invention, of the ammonium salts of formula (XIV) in which:
R₂₂ denotes a methyl or ethyl radical,
x and y are equal to 1;
z is equal to 0 or 1;
r, s and t are equal to 2;
R₂₃ est choisi parmi:
the

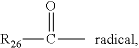

methyl, ethyl or C₁₄-C₂₂ hydrocarbon-based radicals,
a hydrogen atom;
R₂₅ is chosen from:
the

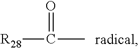

a hydrogen atom;
R₂₄, R₂₆ and R₂₈, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Mention may be made, for example, of the compounds of formula (XIV) such as diacyloxyethyldimethylammonium, diacyloxyethylhydroxyethylmethylammonium, monoacyloxyethyldihydroxyethylmethylammonium, triacyloxy-ethylmethylammonium or monoacyloxyethylhydroxyethyldimethylammonium salts (chloride or methyl sulphate, in particular) and their mixtures. The acyl radicals preferably have from 14 to 18 carbon atoms and originate more particularly from a vegetable oil, such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are, for example, sold under the names Dehyquart® by Henkel, Stepanquat® by Stepan, Noxamium® by Ceca or Rewoquat® WE 18 by Rewo-Witco.

The composition according to the invention preferably contains a mixture of quaternary ammonium mono-, di- and triester salts with a weight majority of diester salts.

Examples of mixtures of ammonium salts that may be used include the mixture containing 15% to 30% by weight of acyloxyethyldihydroxyethyl-methylammonium methyl sulphate, 45% to 60% of diacyloxyethyl-hydroxyethylmethylammonium methyl sulphate and 15% to 30% of triacyloxyethylmethylammonium methyl sulphate, the acyl radicals containing from 14 to 18 carbon atoms and being derived from palm oil that is optionally partially hydrogenated.

Use may also be made of the ammonium salts comprising at least one ester functional group described in U.S. Pat. No. 4,874,554 and U.S. Pat. No. 4,137,180.

Preferably, the cationic surfactants of the invention are chosen from the compounds of formulae (XI) and/or (XIV).

Preference is given, among the cationic surfactants of formulae (XI) to (XIV), on the one hand, to tetraalkylammonium chlorides, such as, for example, dialkyldimethylammonium or alkyltrimethylammonium chlorides in which the alkyl radical comprises approximately from 12 to 22 carbon atoms, in particular behenyltrimethylammonium chloride, distearyldimethylammonium chloride, cetyltrimethylammonium chloride or benzyldimethylstearylammonium chloride, or also, on the other hand, to distearoylethylhydroxyethylmethylammonium methosulphate, dipalm itoylethylhydroxyethylammonium methosulphate or distearoylethyl hydroxyethylammonium methosulphate, or also, finally, to palmitylam idopropyltrimethylammonium chloride or stearamidopropyldimethyl(myristyl acetate)ammonium chloride, sold under the name Ceraphyl® 70 by Van Dyk.

It is preferable, among all the cationic surfactants which can be present in the composition according to the invention, to choose cetyltrimethylammonium, behenyltrimethylammonium, dipalm itoylethylhydroxyethylmethylammonium, distearoylethylhydroxyethylmethylammonium or methyl(C₉-C₁₉)alkyl(C₁₀-C₂₀)alkylamidoethylimidazolium salts, the stearamidopropyldimethylammonium salt and their mixtures.

Preferably, oleocetyldimethylhydroxyethylammonium chloride will be chosen as cationic surfactant.

The cationic surfactants ii) can be present each in a content ranging from 0.01% to 15% by weight, preferably ranging from 0.1% to 10% by weight and more preferably ranging from 0.2% to 5% by weight, with respect to the total weight of the composition of the invention.

Preferably, the cationic polymer(s) (i)/cationic surfactants ratio by weight is less than 1. Better still, it is between 0.05 and 1.

Polysaccharide Thickener

The term “thickener” is understood to mean, within the meaning of the invention, a compound capable, by its presence, of increasing the viscosity of the medium by at least 50 cPs at 25° C. and at a shear rate of 1 s⁻¹. Preferably, the thickening agent, at 1% in water or a 50/50 by weight water/alcohol mixture, exhibits a viscosity at 25° C. and at a shear rate of 1 s⁻¹ of greater than 100 cPs.

These viscosities can be measured using in particular viscometers or rheometers having cone/plate geometry.

Polysaccharide thickeners are polymers which exhibit monosaccharides or disaccharides as base units. The polymers which can be used in the compositions according to the present invention are preferably chosen from guar gums and modified guar gums, celluloses, gellan gum and its derivatives.

Preferably, the polysaccharides of the invention are nonionic.

Guar gums are galactomannans composed of mannose and of galactose.

The term “modified guar gum” is understood to mean, within the meaning of the present patent application, guar gums alkylated by at least one C₁₋₈ alkyl group, guar gums hydroxyalkylated by at least one C₁₋₈ hydroxyalkyl group or guar gums acylated by at least one C₁₋₈ acyl group.

Preferably, they will be hydroxypropylated guar gums, such as that sold under the name Jaguar HP 105 by Rhodia.

Cellulose is a β-1,4-polyacetal of cellobiose, cellobiose being a disaccharide composed of two glucose molecules.

The celluloses which can be used in the compositions according to the present invention are preferably chosen from nonionic celluloses not comprising a hydrophobic chain and nonionic celluloses comprising one or more hydrophobic chains.

Preferably, the ionic celluloses used according to the invention are cellulose ethers. More preferably still, these celluloses are hydroxyalkylcelluloses and in particular hydroxyethylcelluloses or hydropropylcelluloses. They may or may not contain a fatty chain. Among the non-ionic celluloses that do not comprise a fatty chain, mention may be made of hydroxyethylcelluloses, hydropropylcelluloses, and hydroxypropylmethylcelluloses. One particularly suitable hydroxypropylmethylcellulose is Methocel F4M sold by Dow Chemicals (INCI name: Hydroxypropylmethylcellulose).

The celluloses modified with groups comprising one or more nonionic fatty chains that can be used according to the present invention are, in particular:
hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C₈-C₂₂ alkyl groups, such as the product Natrosol Plus Grade 330 CS® (C₁₆ alkyls), sold by Aqualon, corresponding to the INCI name Cetylhydroxyethylcellulose, or the product Bermocoll EHM 100® sold by Berol Nobel,
those modified with alkylphenyl polyalkylene glycol ether groups, such as the product Amercell Polymer HM-1500® (nonylphenyl polyethylene glycol (15) ether) sold by Amerchol that corresponds to the INCI name Nonoxynyl hydroxyethylcellulose.

Gellan gum is a polysaccharide produced by aerobic fermentation of Sphingomonas elodea, more commonly known as Pseudomonas elodea. This linear polysaccharide is composed of the sequence of the following monosaccharides: D-glucose, D-glucuronic acid and L-rhamnose. In the native state, gellan gum is highly acylated.

The gellan gum preferably used in the compositions according to the present invention is an at least partially deacylated gellan gum. This at least partially deacylated gellan gum is obtained by a high-temperature alkaline treatment. A KOH or NaOH solution will, for example, be used.

The purified gellan gum sold under the trade name Kelcogel® by Kelco is suitable for preparing the compositions according to the invention.

The derivatives of gellan gum are all products obtained by carrying out conventional chemical reactions, such as in particular esterifications or addition of a salt of an organic or inorganic acid.

Use is made, as derivative of gellan gum, for example, of welan gum. Welan gum is a gellan gum modified by fermentation by means of Alcaligenes strain ATCC 31 555. Welan gum has a repeating pentasaccharide structure formed of a main chain composed of D-glucose, D-glucuronic acid and L-rhamnose units to which a pendant unit of L-rhamnose or of L-mannose is grafted.

The welan gum sold under the trade name Kelco Crete® by Kelco is suitable for preparing the compositions according to the invention.

Mention may be made, as other saccharide thickening polymers which can be used according to the invention, of starches and their derivatives.

The concentration of polysaccharide thickeners used in the compositions according to the present invention is between 0.05 and 10% by weight, preferably between 0.1 and 5% by weight and more preferably still between 0.5 and 3% by weight, with respect to the total weight of the composition.

Polyol

The polyols which can be used in the composition according to the present invention are compounds composed of a hydrocarbon-based chain optionally interrupted by one or more oxygen atoms and carrying at least two free hydroxyl groups and with a molecular weight of less than 1000.

The hydrocarbon-based chain can be linear or branched and saturated or unsaturated.

Preferably, the hydrocarbon-based chain comprises from 3 to 50 carbon atoms and better still from 3 to 20 carbon atoms.

In an alternative form of the invention, the hydrocarbon-based chain is saturated.

Very particularly, the polyols of the invention comprise 2 or 3 free hydroxyl functional groups.

The free hydroxyl functional groups can be carried by contiguous carbon atoms or the carbon atoms carrying them can be separated by one or more other carbon atoms.

Use is preferably made, among these polyols, of 1,2-propanediol (or propylene glycol), 1,3-propanediol, glycerol, 3-(2-ethylhexyl)oxy-1,2-propanediol (or ethylhexylglycerin), 3-methyl-1,3,5-pentanetriol, 1,2,4-butanetriol, 1,5-pentanediol, 2-methyl-1,3-propanediol, 1,3-butanediol, 3-methyl-1,5-pentanediol, neopentyl glycol (2,2-dimethyl-1,3-propanediol), isoprene glycol (3-methyl-1,3-butanediol) and hexylene glycol (2-methyl-2,4-pentanediol), diethylene glycol, dipropylene glycol, tripropylene glycol or polyethylene glycols of formula H(OCH₂CH₂)ₙOH with n ranging from 4 to 16.

Preferably, the molecular weight (MW) of the polyol is between 75 and 500. More preferably still, the molecular weight is between 90 and 350.

The polyol or polyols can be present in the composition at a content of between 0.1 and 30% by weight, preferably between 0.5 and 20% by weight and more preferably still between 1 and 15% by weight of the total weight of the cosmetic composition.

Medium

The composition according to the invention can additionally comprise one or more conventional additives well known in the art other than the compounds defined above. Mention may be made, as examples of additives which can be used according to the invention, of silanes, anionic surfactants, amphoteric or zwitterionic surfactants, nonionic surfactants, proteins, protein hydrolysates, vitamins, reducing agents, plasticizers, softeners, antifoaming agents, moisturizing agents, pigments, clays, inorganic fillers, UV screening agents, abrasive agents (pumice, apricot kernel powder), inorganic colloids, peptizing agents, solubilizing agents, fragrances, preservatives, pearlescent agents, propellants, inorganic or organic thickeners, antidandruff agents (for example, zinc pyrithione, octopirox, selenium sulphide, ellagic acid and derivatives), agents for combating hair loss or agents for promoting hair regrowth; these additives being other than the compounds defined above.

A person skilled in the art will take care to select the optional additive(s) and the amount thereof such that they do not harm the properties of the compositions of the present invention.

The additive(s) are generally present in the composition according to the invention in an amount ranging from 0 to 20% by weight relative to the total weight of the composition.

The compositions according to the invention can be provided in all the formulation forms conventionally used for a topical application and in particular in the form of aqueous or aqueous/alcoholic solutions, of oil-in-water (O/W), water-in-oil (W/O) or multiple (triple: W/O/W or O/W/O) emulsions, of aqueous gels or of dispersions of a fatty phase in an aqueous phase using spherules, it being possible for these spherules to be polymeric nanoparticles, such as nanospheres and nanocapsules, or lipid vesicles of ionic and/or nonionic type (liposomes, niosomes or oleosomes). These compositions are prepared according to the usual methods.

In addition, the compositions used according to the invention can be more or less fluid and can have the appearance of a white or coloured cream, of an ointment, of a milk, of a lotion, of a serum, of a paste or of a foam. They can optionally be applied to the keratinous substances in the aerosol form. They can also be provided in the solid form and for example in the stick form.

Of course, the person skilled in the art will take care to choose the optional compound or compounds to be added to the composition according to the invention so that the advantageous properties intrinsically attached to the composition in accordance with the invention are not, or not substantially, detrimentally affected by the envisaged addition or additions.

Another subject-matter of the invention is a method for the cosmetic treatment of keratinous substances, in particular keratinous fibres and especially human keratinous fibres, such as the hair, comprising the application of a composition according to the invention to the said keratinous substances and in particular the keratinous fibres. After an optional leave-in time preferably ranging from 1 to 30 minutes, the composition is subsequently preferably rinsed out with water. Preferably, the said method is intended for the conditioning of the said keratinous fibres.

According to a specific form of the invention, the method for the treatment of keratinous fibres can comprise a stage of heating the keratinous fibres during or after application of the composition according to the invention.

The keratinous fibres can, for example, be heated by means of an iron, of a liquid water/steam mixture or of a drying hood or hairdryer.

The application of the composition of the invention can also be preceded or followed by the application of an acid composition (for example, pH 2 to 6) or basic composition (for example, pH 8 to 12).

Another subject-matter of the invention is the use of a composition according to the invention for caring for keratinous substances, in particular keratinous fibres and especially human keratinous fibres, such as the hair.

The examples which follow are intended to illustrate the invention without, however, exhibiting a limiting nature.

### EXAMPLE 1

### Leave-in Serum

| | Commercial |
|---|---|
| Chemical name | product as is in |
| (Commercial reference) | grams |
| Ethylhexylglycerin | 0.4 |
| (Sensiva SC 50 from Schulke & Mayr) | |
| Hydroxyethylcellulose | 1 |
| (Natrosol 250 MR from Aqualon (Hercules)) | |
| Cetrimonium chloride | 0.5 |
| (Dehyquart A OR from Cognis) | |
| Glycerol | 5 |
| (Refined glycerine from Godrej Industries) | |
| PEG-40 Hydrogenated castor oil | 1 |
| (Cremophor CO 40 from BASF) | |
| Hydroxyethyl oleyl dimonium chloride | 0.75 |
| (Chimexane CL from Chimex) | |
| Behentrimonium chloride | 0.5 |
| (Genamin KDMP from Clariant) | |
| Polyquaternium-6 | 0.9 |
| (Merquat 100 from Nalco) | |
| Preservatives | q.s. |
| Fragrance | q.s. |
| Water | q.s. for 100 g |

This serum is clear. Applied to clean hair, it confers thereon, after drying, sheen, smoothness and softness. The hair is easily disentangled.

### EXAMPLE 2

### Leave-in Serum

| | Commercial |
|---|---|
| Chemical name | product as is in |
| (Commercial reference) | grams |
| Ethylhexylglycerin | 0.4 |
| (Sensiva SC 50 from Schulke & Mayr) | |
| Hydroxyethylcellulose | 1 |
| (Natrosol 250 MR from Aqualon (Hercules)) | |
| Glycerol | 5 |
| (Refined glycerine from Godrej Industries) | |
| PEG-40 Hydrogenated castor oil | 1 |
| (Cremophor CO 40 from BASF) | |
| PEG-15 Cocopolyamine | 2 |
| (Dehyquart H 81 from Cognis) | |
| Behentrimonium chloride | 1 |
| (Genamin KDMP from Clariant) | |
| Hydroxyethyl oleyl dimonium chloride | 0.75 |
| (Chimexane CL from Chimex) | |
| Polyquaternium-6 | 0.9 |
| (Merquat 100 from Nalco) | |
| Preservatives | q.s. |
| Fragrance | q.s. |
| Water | q.s. for 100 g |

### EXAMPLE 3

### Leave-in Serum

| Chemical name | % as is as |
|---|---|
| (Commercial reference) | weight/weight |
| Ethylhexylglycerin | 0.4 |
| (Sensiva SC 50 from Schulke & Mayr) | |
| Hydroxyethylcellulose | 1 |
| (Natrosol 250 MR from Aqualon (Hercules)) | |
| Divinyldimethicone/dimethicone copolymer (and) | 1 |
| cetrimonium chloride (and) hydroxyethylcellulose | |
| (Dow Corning 2 1997 Cationic Emulsion from | |
| Dow Corning) | |
| Glycerol | 5 |
| (Refined glycerine from Godrej Industries) | |
| PEG-40 Hydrogenated castor oil | 1 |
| (Cremophor CO 40 from BASF) | |
| Hydroxyethyl oleyl dimonium chloride | 0.75 |
| (Chimexane CL from Chimex) | |
| Behentrimonium chloride | 1 |
| (Genamin KDMP from Clariant) | |
| Polyquaternium-6 | 0.9 |
| (Merquat 100 from Nalco) | |
| Preservatives | q.s. |
| Fragrance | q.s. |
| Water | q.s. for 100 g |

1-13. (canceled)

14. A cosmetic composition comprising:
(i) one or more cationic polymers, wherein at least one cationic polymer is a dimethyldiallylammonium chloride homopolymer;
(ii) at least two cationic surfactants;
(iii) at least one polysaccharide thickener; and
(iv) at least one polyol with a molecular weight of less than about 1000.

15. The composition according to claim 14, wherein the at least one cationic polymer is present in an amount ranging from about 0.01% to about 15% by weight with respect to the total weight of the cosmetic composition.

16. The composition according to claim 14, wherein the at least one cationic polymer is present in an amount ranging from about 0.1% to about 10% by weight with respect to the total weight of the cosmetic composition.

17. The composition according to claim 14, wherein the at least one cationic polymer is present in an amount ranging from about 0.2% to about 5% by weight with respect to the total weight of the cosmetic composition.

18. The composition according to claim 14, wherein the at least two cationic surfactants are independently chosen from:
the quaternary ammonium salts corresponding to the following general formula (I): 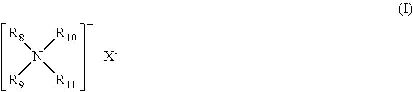
in which the R₈ to R₁₁ radicals, which are identical or different, represent a linear or branched aliphatic radical comprising from 1 to 30 carbon atoms and which can comprise heteroatoms, or else an aromatic radical, at least one of the R₈ to R₁₁ radicals comprising from 8 to 30 carbon atoms;
imidazoline quaternary ammonium salts corresponding to the following formula (II): 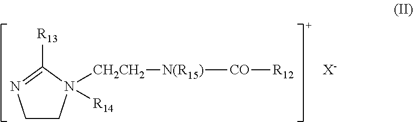
in which R₁₂ represents an alkenyl or alkyl radical comprising from 8 to 30 carbon atoms, R₁₃ represents a hydrogen atom, a C₁-C₄ alkyl radical or an alkenyl or alkyl radical comprising from 8 to 30 carbon atoms, R₁₄ represents a C₁-C₄ alkyl radical, R₁₅ represents a hydrogen atom or a C₁-C₄ alkyl radical and X⁻ is an anion chosen from the group of the halides, phosphates, acetates, lactates, alkyl sulphates, or alkyl- or alkylarylsulphonates;
di- or triquaternary ammonium salts of formula (XIII): 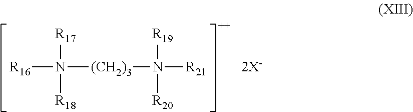
in which R₁₆ denotes an alkyl radical comprising approximately from 16 to 30 carbon atoms which is optionally hydroxylated and/or interrupted by one or more oxygen atoms, R₁₇ is chosen from hydrogen or an alkyl radical comprising from 1 to 4 carbon atoms or an (R₁₆ₐ)(R₁₇ₐ)(R₁₈ₐ)N⁺(CH₂)₃— group, R₁₆ₐ, R₁₇ₐ, R₁₈ₐ, R₁₈, R₁₉, R₂₀ and R₂₁, which are identical or different, are chosen from hydrogen or an alkyl radical comprising from 1 to 4 carbon atoms and X⁻ is an anion chosen from the group of the halides, acetates, phosphates, nitrates and methyl sulphates;
quaternary ammonium salts comprising at least one ester functional group of following formula (XIV): 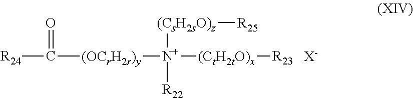
in which:
R₂₂ is chosen from C₁-C₆ alkyl and C₁-C₆ hydroxyalkyl or dihydroxyalkyl radicals;
R₂₃ is chosen from:
the 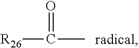
radicals R₂₇, which are linear or branched, saturated or unsaturated C₁-C₂₂ hydrocarbon-based radicals,
a hydrogen atom,
R₂₅ is chosen from:
the 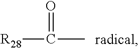
radicals R₂₉, which are linear or branched, saturated or unsaturated C₁-C₆ hydrocarbon-based radicals,
a hydrogen atom,
R₂₄, R₂₆ and R₂₈, which may be identical or different, are chosen from linear or branched, saturated or unsaturated C₇-C₂₁ hydrocarbon-based radicals;
r, s and t, which may be identical or different, are integers ranging from 2 to 6;
y is an integer ranging from 1 to 10;
x and z, which may be identical or different, are integers ranging from 0 to 10;
X⁻ is a simple or complex, organic or inorganic anion;
with the proviso that the sum x+y+z is from 1 to 15, that when x is 0, then R₂₃ denotes R₂₇ and that when z is 0, then R₂₅ denotes R₂₉.

19. The composition according to claim 18, wherein the at least two cationic surfactants are independently chosen from the compounds of formulae (XIII) and (XIV).

20. The composition according to claim 14, wherein the at least two cationic surfactants are each present in an amount ranging from about 0.01% to about 15% by weight with respect to the total weight of the cosmetic composition.

21. The composition according to claim 14, wherein the at least two cationic surfactants are each present in an amount ranging from about 0.1% to about 10% by weight with respect to the total weight of the cosmetic composition.

22. The composition according to claim 14, wherein the at least two cationic surfactants are each present in an amount ranging from about 0.2% to about 5% by weight with respect to the total weight of the cosmetic composition.

23. The cosmetic composition according to claim 14, comprising at least one polysaccharide thickener chosen from nonionic polysaccharide thickeners.

24. The cosmetic composition according to claim 14, comprising at least one polysaccharide thickener chosen from celluloses.

25. The cosmetic composition according to claim 14, comprising at least one polysaccharide thickener chosen from cellulose ethers.

26. The cosmetic composition according to claim 14, comprising at least one polyol chosen from compounds composed of a hydrocarbon-based chain comprising from 3 to 50 carbon atoms and optionally interrupted by one or more oxygen atoms and carrying at least two free hydroxyl groups.

27. The cosmetic composition according to claim 14, comprising at least one polyol chosen from 1,2-propanediol (or propylene glycol); 1,3-propanediol; glycerol; 3-(2-ethylhexyl)oxy-1,2-propanediol (or ethylhexylglycerin); 3-methyl-1,3,5-pentanetriol; 1,2,4-butanetriol; 1,5-pentanediol, 2-methyl-1,3-propanediol; 1,3-butanediol; 3-methyl-1,5-pentanediol; neopentyl glycol (2,2-dimethyl-1,3-propanediol); isoprene glycol (3-methyl-1,3-butanediol) and hexylene glycol (2-methyl-2,4-pentanediol); diethylene glycol; dipropylene glycol; and tripropylene glycol or polyethylene glycols of formula H(OCH₂CH₂)ₙOH with n ranging from 4 to 16.

28. The cosmetic composition according to claim 14, wherein the molecular weight (MW) of the at least one polyol ranges from about 75 to 500.

29. The cosmetic composition according to claim 14, wherein the at least one polyol is present in the composition in an amount ranging from about 0.1% to about 30% by weight with respect to the total weight of the cosmetic composition.

30. The cosmetic composition according to claim 14, wherein the at least one polyol is present in the composition in an amount ranging from about 0.5% to about 20% by weight with respect to the total weight of the cosmetic composition.

31. The cosmetic composition according to claim 14, wherein the at least one polyol is present in the composition in an amount ranging from about 1% to about 15% by weight with respect to the total weight of the cosmetic composition.

32. A method for the nontherapeutic cosmetic treatment of keratinous substances comprising applying to said substances a cosmetic composition comprising:
(i) one or more cationic polymers, wherein at least one cationic polymer is a dimethyldiallylammonium chloride homopolymer;
(ii) at least two cationic surfactants;
(iii) at least one polysaccharide thickener; and
(iv) at least one polyol with a molecular weight of less than about 1000.
